(19) United States
(12) Patent Application Publication
(10) Pub. No.: US 2011/0165647 A1
(43) Pub. Date: Jul. 7, 2011

(54) NANOPARTICLE CONJUGATES

(76) Inventors: David G. Fernig, Merseyside (GB); Laurence Duchesne, Paris (FR)

(21) Appl. No.: 12/866,012

(22) PCT Filed: Feb. 4, 2009
(86) PCT No.: PCT/GB2009/050107

§ 371 Date: Feb. 8, 2011

(30) Foreign Application Priority Data
Feb. 4, 2008 (GB) 0801988.7
Sep. 2, 2008 (GB) 0815949.3

(51) Int. Cl. C12N 9/96; C07K 7/06; C07K 5/083; C07K 5/103; C07K 7/08; C07K 19/00; C07K 14/77; B82Y 30/00; B82Y 40/00

(52) U.S. Cl. 435/188; 530/330; 530/329; 530/331; 530/328; 530/326; 530/391.1; 530/367; 977/773; 977/774; 977/838; 977/810; 977/896

(57) ABSTRACT

A nanoparticle conjugate comprising a nanoparticle having one or more peptide-ol compounds and one or more polyethylene glycol (PEG) compounds attached thereto. A method of producing the nanoparticle conjugate is also described.

The present invention relates to a nanoparticle conjugates

The numerous applications of metal and semi-conductor nanoparticles require the protection of the metal and semi-conductor core from the environment for three reasons. Firstly, the metal and metal-derived core is not inert, but will interact with many molecules. Therefore, a naked nanoparticle will suffer from a wide range of undesired non-specific interactions. Secondly, apart from gold, other nanoparticle materials suffer from chemical instability in aqueous environment in the presence of atmospheric oxygen. For example, silver readily oxidises, so for most metals and semi-conductors, protection from the environment is essential to preserve the nanoparticle core, whose properties are the basis of detection and hence the envisaged application. Thirdly, the metal and metal-derived nanoparticles are readily aggregated by electrolytes at concentrations typical of biological samples.

The design of ligand shell or matrix systems presents major challenges, which have been reviewed recently (Doty, R. C., et. al., (2004) CMLS, Cell. Mol. Life. Sci., 1843-1849).

Since thiols form strong covalent bonds with noble metal nanoparticles, existing ligand shells often possess one or more thiols and include alkyl thiols and derivatives, e.g., mercaptoundecanoic acid (MUA), lipoate, thiolated dextrans and polyethylene glycols.

There are two classes of ligand shell system. One class consists of large polymers that coat the nanoparticle that may possess a negative charge. These polymers, e.g., thiolated dextrans, various block copolymers, do produce reasonably stable nanoparticles. Moreover, when the bonding of the ligand to the nanoparticle is relatively weak, e.g., thiol to semi-conductor or transition metal, the presence of multiple attachment sites for each polymer molecule is thought to impart increased stability to the nanoparticle. However, the thickness of these ligand shells cannot be controlled and the hydrodynamic radius of the nanoparticle is augmented considerably by the polymer ligand shell. For example, semi conductor nanoparticles (quantum dots) are typically protected by a block copolymer ligand shell, which increases the hydrodynamic radius of the material to 15 nm to 20 nm. Moreover, the polymers are known to form local microenvironments that can adsorb biological macromolecules and stoichiometric coupling of macromolecules to functional ligands is difficult and often impossible. For example, functions grafted onto the block copolymer may not be readily accessible to the environment should they be present at some depth. The second class consist of small molecules that self-organise on the surface of the nanoparticle to produce a tight “skin” of defined thickness. These are often dependent on the presence of a charged group at the end of the molecule that is on the “outside” of the ligand shell and so interacting with the solvent. This charge allows for solubility in water and charge repulsion is thought to be significant in preventing aggregation of the nanoparticles. Another consideration is the packing and depth of the self-assembled monolayer. Very small molecules such as lipoate, cysteine, and glutathione (ECG) are generally too small to provide good stabilisation in biological environments, whereas longer molecules, e.g., thiolated oligonucleotides, mercaptoundecanoic acid provide a reasonable degree of stabilisation. Importantly, the defined dimensions of these ligand shells means that functions grafted onto the ligand shell are predictably exposed to the environment.

One embodiment of this second class of ligand shell is a peptide based system (Levy, R., et. al., (2004) J. Am. Chem. Soc., 126, (32), 10076-10084). This peptide based system has also been described in WO2005/029076, in which a nanoparticle conjugate comprising a nanoparticle conjugated to a plurality of peptides of a substantially similar amino acid sequence having peptides conjugated to the nanoparticle by means of a Cysteine (C) residue and the nanoparticle conjugate further comprising a ligand attached to the peptides. A second embodiment is the thiolated alkyl PEG system (Doty, R. C., Tshikhudo, T. R., Brust, M., Fernig, D. G. (2005). Extremely stable water-soluble Ag nanoparticles. Chem. Mater. 17: 4630-4635).

An important issue with this second class of ligand is that stabilisation of the nanoparticle is thought to depend on both the strength of the interaction of the ligand with the nanoparticle and on the lateral interactions of the ligands within the self-assembled monolayer. Consequently, this approach has largely been restricted to thiolated ligands and noble metal nanoparticles, since the thiol groups bonds strongly to these metals.

It is, therefore, an object of the present invention to provide a metal/metal derived and semi-conductor nanoparticle conjugates having increased stability under a range of conditions.

In accordance with the present invention, there is provided a nanoparticle conjugate comprising a nanoparticle having one or more compounds attached thereto, wherein at least one of the compounds comprises at least one ethylene glycol unit.

The nanoparticle conjugate of the present invention has been found to have unexpected properties that provide substantial advantages over other prior art ligand shell systems. Such nanoparticle conjugates have been found to have extremely stable characteristics under a range of conditions.

Preferably, the nanoparticle conjugate comprises a nanoparticle having one or more peptide-ol and/or one or more polyethylene glycol compound and/or one or more peptide-ethylene glycol compounds and/or one or more thiol alkane polyethylene glycol (HSPEG) compounds attached thereto.

The term “peptide-ol”, should be understood to be a peptide-ol which has an alcohol (CH₂OH) group in place of the carboxyl (COOH) group at the C-terminus. The C-terminal moiety is, therefore an amino alcohol, rather than an amino acid, though the synthetic route of the peptidol may involve conversion from an amino acid (or other convenient synthon) to the alcohol.

The term “peptide-ethylene glycol” should be understood to be a peptide which has one or more ethylene glycol units in place of the carboxyl (COOH) group at the C-terminus.

The stability imparted to nanoparticles by a mixture of the peptide-ol and PEG compounds greatly exceeds that of using the individual components alone. The stability imparted to nanoparticles by peptide-ethylene glycol is also remarkable. Unexpectedly, the thiol-containing ligands impart great stability to semiconductor nanoparticles, despite the thiol bonding relatively weakly to these materials. The termini of the peptide-ol and PEG compounds exposed to solvent are uncharged and so result in a nanoparticle that is polar, but which does not carry charge. Despite the absence of charge and hence repulsive forces, the nanoparticles are exceptionally stable with respect to their aggregation, their non-specific adsorption to a wide variety of substances and to ligand-exchange.

In the case of mixtures of peptide-ol and PEG compounds (“mixed matrix”) the components of the mixed matrix ligand shell are thought to segregate on the surface of the nanoparticles (Jackson A M, Hu Y, Silva P J, Stellacci F. From homoligand- to mixed-matrix ligand shell-monolayer-protected metal nanoparticles: a scanning tunneling microscopy investigation. J Am Chem. Soc. 2006; 128:11135-49; Duchesne, L., Wells, G., Fernig, D. G., Harris, S. and Levy, R. (2008). Supramolecular domains in mixed peptide self-assembled monolayers on gold nanoparticles. ChemBiochem. 9: 2127-2134), similarly to the well-known phase separation of matrix ligands in mixed self-assembled monolayers on flat metal surfaces and functional groups can be incorporated into one or more of the components, which allows for a combinatorially complex presentation of functions on the nanoparticle surface with a degree of control over their spatial distribution.

The one or more PEG compounds and the one or more peptide-ol compounds preferably form nano domains on the surface of the nanoparticle of predominantly peptide-ol compounds or PEG compounds. As such, it is possible to have more than one functionalised domain, wherein the functionalised domains have different activity/function and which operate separately. More preferably, the compounds form nanodomains exclusively formed from one or more PEG compound or one or more peptide-ol compound.

The peptide-ol, PEG, peptide, alkane thiol PEG and/or the peptide ethylene glycol compound may comprise one or more thiols, as embodied by cysteine residues. Preferably, the peptide-ol(s), PEG, peptide, alkane thiol PEG and/or the peptide ethylene glycol and/or peptide ethylene glycol compound is attached to the nanoparticle by means of a thiol, as embodied by one or more cysteine residues. The one or more cysteine residues may be located at one end of the peptide-ol(s), PEG, peptide, alkane thiol PEG and/or the peptide ethylene glycol compound and the cysteine residue may be attached to the nanoparticle by means of its thiol and/or amino group or carboxylic acid group.

The peptide-ol and/or peptide ethylene glycol compound and the alkyl chain of the PEG compound are preferably substantially the same length.

The one or more peptide-ol, PEG, peptide, alkane thiol PEG and/or the peptide ethylene glycol compounds may comprise at least two amino acids. Preferably, the one or more compound comprises 2 to 30 amino acids. More preferably, the one or more compound comprises 2 to 20, and even more preferably 2 to 10 amino acids. More preferably still, the compound may comprise 4 to 6 amino acids. Most preferably, the compound comprises 5 amino acids.

It is preferred that the peptide-ol and/or peptide ethylene glycol compound is a pentapeptidol or a pentapeptide ethylene glycol. Longer or shorter peptides may also be accommodated, in the cases where the peptide is mixed with a thiol alkane PEG preferably such that the length of the peptide chain (˜0.35 nm per amino acid in extended conformation or beta strand conformation, 0.15 nm per amino acid in alpha helical conformation) is similar to that of the alkyl chain of the thiolated PEG, where the carbon-carbon bonds are of the order of 0.1±0.05 nm. For example, for a thiolated PEG with 11 carbons between the thiol and the first PEG unit, a peptidol of extended conformation of about four to six amino acids is preferred.

If the amino acid attached to the nanoparticle is designated as amino acid #1=C to amino acid #5=X(ol) the peptide-ol and/or peptide ethylene glycol of the present invention preferably comprise the following:

Amino acid #1 (the N-terminal aminoacid) is preferably cysteine or an unnatural aminoacid or thiol with one or more thiol groups;

Amino acid #2 is preferably an amino acid with a hydrophobic side chain (e.g., alanine, leucine, valine), including a cysteine; an amino acid with a polar side chain may also be accommodated at this position, for example serine.

Amino acid #3 is preferably an amino acid with a hydrophobic side chain, for example valine, leucine. A polar amino acid may also be accommodated at this position, for example serine, threonine, asparagine, apartate.

Amino acid #4 is preferably an amino acid with a hydrophobic or a polar side chain, for example valine, asparagine or serine.

The terminal amino acid, #5 in the preferred embodiments, has an alcohol rather than a carboxylic acid, so is in fact an amino alcohol. The amino acid side chain may be hydrophobic or have a polar side chain. Examples include: valine, asparagine, tyrosine, threonine or serine.

The direction of the peptide-ol and/or peptide ethylene glycol may be reversed such that the amino acid attached to the nanoparticle is the C-terminal amino acid and in this case, since the convention for numbering peptides is always N-terminus to C-terminus, if the C-terminal amino acid attached to the nanoparticle is designated as amino acid #5=C, the N-terminal amino acid #1 X(ol) which is in contact with the solvent is the N-terminal amino acid having its amino group replaced by an alcohol, so in fact being a carboxylic acid alcohol or replaced by ethylene glycol units, so in fact being a carboxylic acid ethylene glycol.

Therefore, the present invention provides for a nanoparticle conjugate that has a greatly increased stability in a number of biological and chemical environments. The configuration of the nanoparticle conjugate resembles a protein which may additionally have a “sticky” core (containing for example, an inorganic metallic or semiconductor material) that is hidden by an organised surface (provided by the peptide-ol and PEG compounds) that can therefore be tailored to suit the needs of a given application. It is believed that the secondary structure (alpha helix, beta strand, H-bonding) of the peptide moiety of the peptide-ol and/or peptide ethylene glycol assists in nanoparticle stabilisation. Indeed, peptide-ols and/or peptide ethylene glycol that form beta strands are preferred as the strand formation allows high packing densities of peptide-ols and/or peptide ethylene glycol to nanoparticles to be achieved.

Preferably, the Cysteine (C) residue is conjugated to the nanoparticle by means of its thiol and the amino group or in the case of a reversed sequence, by means of its thiol and the carboxyl group. The exact choice of amino acid sequence will be governed by amino acids that allow close packing on the nanoparticle surface and this in turn will be dictated by the curvature of the nanoparticle amongst other things. The core matrix peptide-ol and/or peptide ethylene glycol may have the general sequence of CXₙ₋₁ X(ol) or (ol) XXₙ₋₁C, where ‘ol’ refers to the terminal alcohol or ethylene glycol units of the peptide. A small number (1-10) of recognition functions may be incorporated into the core matrix ligand shell as functionalised peptide-ol, peptide ethylene glycol, peptides or thiolated alkane PEGs, where n is a number from 1 to 11.

The number of functionalised peptide-ol, peptide ethylene glycol, peptides or thiolated alkane PEGs present on a 10 nm nanoparticle is preferably between 1 and 20, more preferably 1 and 10.

Preferably the percentage of functionalised peptide-ol, peptide ethylene glycol, peptides or thiolated alkane PEGs present on a nanoparticle's surface as a percentage of the total number of conjugated compounds is less than 10%, more preferably 5-10%.

Additional peptide-ols and/or peptide ethylene glycol with different sequences may be incorporated to increase the number of matrix ligand nanodomains of the nanoparticle surface. Functional ligands are incorporated in one or more peptide-ols and/or peptide ethylene glycol as CXn Xn′(ligand)X(ol) (Xn′=any aminoacid used as a spacer for ligand; ‘ol’ terminal alcohol or ethylene glycol units) to give complete freedom to perform function to ligand, CCXn(ligand)X(ol), CXn(ligand)Xₙ₋₁ X(ol) or CCXₙ₋₁ (ligand)Xₙ₋₁ X(ol), where X denotes any amino acid residue, X(ol) is any amino acid with the carboxyl group replaced by an alcohol group or ethylene glycol units and n denotes any length of amino acid residues. The equivalent reverse sequences are also included, such as the equivalent reverse sequences (ol)X(ligand)Xₙ₋₁ C, where (ol)X is an amino acid with the amino group replaced by a hydroxyl group or ethylene glycol units. Preferably, the peptide-ol sequence independent of the ligand has the sequence H₂N-Cysteine-Alanine-Leucine-Asparagine-Asparaginol (CALNN(ol)), H₂N-Cysteine-Cysteine-Alanine-Leucine-Asparagine-Asparaginol-(CCALNN(ol)), H₂N-Cysteine-Valine-Valine-Valine-Threoninol-(CVVVT(ol)), H₂N-Cysteine-Cysteine-Valine-Valine-Valine-Threoninol (CCVVVT(ol)), H₂N-Cysteine-Serine-Serine-Serine-Serinol, (CSSSS(ol)), H₂N-Cysteine-Cysteine-Serine-Serine-Serine-Serinol, (CCSSSS(ol)) or the reverse of the above sequences where the N-terminal amino acid having its amino group replaced by an alcohol, so in fact being a carboxylic acid alcohol. In the case of peptide ethylene glycol the identical sequences would have one or more ethylene glycol units in place of the terminal “ol”. In addition, when the relative ratio of peptide-ol to peptide-ol with ligand is low (10% or less), then ligand may be incorporated on a conventional peptide with the carboxylic acid group, e.g., CXₙ₋₁ (ligand), CCXₙ₋₁ (ligand), CXₙ₋₁ (ligand)Xn or CCXₙ₋₁ (ligand)Xn, where the carboxy terminal amino acid is any amino acid with a carboxylic acid, since the added charge is modest and does not disrupt the stability of the nanoparticles.

Incorporation of aminoacids with large side chains such as tryptophan into the peptide-ol and/or peptide ethylene glycol (so X=tryptophan) will result in fewer peptide-ols/unit area and/or peptide ethylene glycol/unit area compared to peptide-ols with amino acids possessing small side chains such as alanine (so X=alanine). The ratio of peptide-ol:thiolated PEG will alter the number of peptide-ols/unit area. It is preferred that there are approximately in the range of 0.2-5, peptide-ol preferably, 0.3-3.2 and per nm² of the nanoparticle. Furthermore, the density of peptide-ol and PEG compounds on a nanoparticle may differ for larger nanoparticles. Preferably, the density of peptide-ol and PEG compounds per nanoparticle will be as high as possible in order to obtain a close packed arrangement.

In an experiment with 9.6 nm gold nanoparticles capped with 7:3 CVVVT(ol) (where of is alcohol) to thiol-undecane(PEG)₃ (HS-C11EG₃) and a total initial concentration of these ligands of 2 mM may have between 450 and 1900, preferably 400-1900 and 1000 CVVVT(ol) peptide-ols per nanoparticle, although potentially this figure could be within the range of −45-4000 peptide-ols per nanoparticle, preferably 70-1500. On this basis a substantially spherical nanoparticle with a diameter of 9.6 nm would have an approximate surface area of 290 nm², which equates to allowing between approximately 1.1-3.6 CVVVT(ol) peptide-ols per nm² of the nanoparticle, and potentially this figure could be in the range 0.15-5; the number of peptide-ols can be tailored for different applications and it will also be dependent not only upon the total surface area of the nanoparticle, but also its curvature. The bulkiness of the side chains on the amino acids of the peptide-ol will affect the number of peptide-ols per unit area.

The PEG compound will preferably be a thiolated alkyl ethylene glycol or a peptide ethylene glycol. It is preferred that the length of the alkyl chain is substantially similar to the length of the peptide-ol compound. The number of ethylene glycol units can be as low as 2 and as high as 100, though 2-10 is preferred and 2-6 provides a lower hydrodynamic radius to the nanoparticle. The one or more peptide-ol compounds may be selected from one of the following: CVT-ol, CVVT-ol, CVVVT-ol, CSSSS-ol CALNN-ol, CAVLT-ol, CAVYT-ol, CAVLY-ol, CAVVY-ol, CVLLY-ol, CVLIT-ol, CVDVT-ol, CVKVT-ol, CFFFT-ol, CVVVVTol, CCVVVVT-ol, CALVVVVT-ol, or a mixture thereof.

A plurality of peptide-ol compounds and a plurality of PEG compounds may be attached to the surface of the nanoparticle so as to provide a shell. It will therefore be apparent to one skilled in the art that such a shell will “shield” the nanoparticle core throughout a number of cytological and biological environments and allow the nanoparticle to remain extremely stable.

It is preferred that the peptide-ol compounds and PEG compounds are present in ratios between 95:5 and 5:95 (mole/mole). More preferably, the peptide-ol compounds and PEG compounds are present in ratios between 10:90 and 90:10:1. Even more preferably, the peptide-ol compounds and PEG compounds are present in ratios between 80:20 and 40:60. Most preferably, the peptide-ol compounds and PEG compounds are present in a ratio of 70:30.

In one embodiment, where the one or more compound comprises peptide ethylene glycol (peptideEG or PEPEG), the nanoparticle preferably comprises a ligand matrix shell consisting or consisting essentially of peptide ethylene glycol.

In one embodiment, where the one or more compound comprises thiolated alkane PEG and the nanoparticle comprises a quantum dot, the nanoparticle preferably comprises a ligand matrix shell consisting or consisting essentially of thiolated alkane PEG.

In another embodiment, where the one or more compound comprises alkane thiol PEG, the nanoparticle preferably comprises a matrix ligand shell consisting or consisting essentially of alkane thiol PEG.

In one embodiment, the compound preferably has the formula:

SH—(CH2)n-EGx
Where:
n=1 to 20;
EG=ethylene glycol unit; and
x=1 to 10
   More preferably, n=5 to 15 and x=2 to 6. Even more preferably, n=11 and x=4.

In another embodiment, the one or more compound preferably has the formula: CVVVT-EGn-ol

Where EG=ethylene glycol unit; and

n=1 to 10.

More preferably, n=5 to 15 and x=2 to 6. Even more preferably, n=11 and x=4.

The nanoparticle conjugate may further comprise one or more functional ligands attached to the peptide-ol compounds and/or the PEG compounds and/or a conventional peptide and/or the alkane thiol PEG and/or the peptide thylene glycol. The term functional ligand encompasses peptide-ol(s), PEG, peptide, alkane thiol PEG and/or the peptide ethylene glycol with an extension/ligand(s) carrying a function, including elthylene glycol units and PEG(s) carrying a function(s) on the ethylene glycol units. Such a ligand may be selected from any number of different molecules that are capable of binding or reacting with other molecules in order to either adhere the nanoparticle to a particular site which may be for identification of a certain molecule within a sample or to hold a molecule for later purification or to achieve a chemical transformation. Furthermore, the ligand may also be used to direct the nanoparticle to a certain site, for example to a cell expressing a certain epitope in order to deliver a pharmaceutical compound. Preferably, the ligand may be selected from one or more of the following: nucleic acid, an antibody or part of an antibody, a peptide-ol, a peptide, a protein, a receptor or a target molecule, an enzyme substrate, a saccharide, a polysaccharide and a lipid.

The nanoparticle conjugate may further comprise an identification means attached to the peptide-ol(s), PEG compound, peptide, alkane thiol PEG and/or the peptide ethylene glycol. Alternatively, an identification means may be attached to one or more of the ligands. An “identification means” should be taken to include functional groups also. An additional sequence of amino acid residues may also be disposed between the ligand and the identification means and/or functionalised group and/or the ligand or identification means or functional group. Therefore, if desired, a “spacer” element may be placed between the core peptide-ol sequence and the ligand and/or placed between the ligand and the identification means/functional group.

The nanoparticle conjugate may comprise different subgroups of peptide-ols. The different ligands and optionally different identification means/functional groups may be attached to different subgroups of the peptide-ols and/or PEG compounds. Synthetic peptide-ol chemistry, which is automated and extremely versatile, can be used to introduce identification means and/or functional groups (such as tags) into the peptide-ols. The identification means and/or functional groups need not be natural and may be unnatural (the latter including D-amino acids and amino acids with synthetic side chains possessing unique chemical reactivities, for example).

The nanoparticle conjugate may be capable of being conjugated to at least one other nanoparticle conjugate or conjugated to a plurality of other nanoparticle conjugates to form nanoparticle conjugate assemblies. Such assemblies can be used for probing or diagnostic tools for identifying a number of variables, such as a number of different antigens on a cell surface, or as a means to amplify the signal by increasing the number of nanoparticles associated with a primary nanoparticle-analyte interaction and creating novel substrates for example.

The nanoparticle may be produced from one of the following materials; a metal material, a magnetic material or a semi-conducting material. The nanoparticle may be produced from a gold, silver, cobalt, nickel, platinum, cadmium selenide or zinc sulphide or other materials used to produce “quantum dots” or similar nanoparticles and colloids.

Magnetic nanoparticles have many applications in biomedicine, such as contrast enhancement agents for magnetic resonance imaging, targeted therapeutic drug delivery and hyperthermia treatment for cancers (Berry, C. C. and Curtis A. S. G., (2003) J. Phys. D: Appl. Phys. 36: R189-206 and Parkhurst, Q. A. et al., (2003) J. Phys. D: Appl. Phys. 36: R167-181). Magnetic immunoassay techniques have also been developed in which the magnetic field generated by the magnetically labeled targets is detected directly with a sensitive magnetometer (Chemla, Y. R., et al., (2000) P. Natl. Acad. Sci. USA. 97: 14268-14272) and such techniques may be used in accordance with the present invention.

It will be apparent to one skilled in the art that should magnetic nanoparticles be employed in accordance with the present invention, such nanoparticles will preferably possess large saturation magnetisation and high magnetic susceptibility so that they respond strongly (Sensitive) to small external/applied magnetic fields or the signal of a magnetic sensor; but weakly respond to other forces such as gravity, Brownian motion, viscosity, van der Waals interactions. Furthermore, the nanoparticles may also be superparamagnetic at room temperature (i.e. the magnetic moment fluctuates freely in the absence of a magnetic field and thus it behaves as non-magnetic) so as to avoid the aggregation of particles. The full exploitation of these properties of magnetic nanoparticles may require size or shape monodispersity and complete or substantially complete stability in biological environments, including, stability in air and aqueous solutions.

The identification means may be selected from a number of molecules and/or compounds that are commonly used for identifying or “tagging” the binding of a ligand to a target molecule. It will be appreciated that molecules and compounds which have yet to be developed may also be employed as an identification means.

The identification means and/or functionalised group and/or ligand may be selected from one or more of the following: biotin and/or avidin, streptavidin, streptactin, Histidine tags, NTA or similar chelator, radio active labels, antigens, epitopes or parts of epitopes, antibodies, fluorochromes, nucleic acids, recognition sequences, enzymes, antibodies, peptides peptide-ols, proteins, receptors or a target molecules, saccharides, polysaccharides and lipids. The identification means and/or functional group may comprise heparan sulphate or heparin and such a nanoparticle conjugate may be conjugated with a mercury adduct or through the polysaccharide's reducing end.

The nanoparticle may further comprise a compound or part of a compound of a pharmaceutically active salt. Therefore the delivery of therapeutic compounds can be directed to different cells or cytological constituents. The provision of part of a pharmaceutically active salt may allow the two-step approach of pro-drug therapy to be utilised. Preferably, the nanoparticle has a diameter in the range of 1-100 nm.

The nanoparticle conjugates may have a wide area of application, for example they may be employed in producing diagnostic assays, separating and/or purifying proteins, or producing therapeutic agents. The nanoparticle conjugate may be used in conjunction with any of the following techniques: chromatography, ELISA, lyophilisation, FISH, ISH, SDS PAGE, flow cytometry, immunohistochemistry, protein purification, western blotting, cytogenetic analysis, molecular interaction assays, histochemistry on fixed and living cells/tissue, electron microscopy, photothermal microscopy, magnetic resonance imaging and high throughput screening.

The nanoparticle conjugates of the present invention have a number of advantages including:

a) High stability/no aggregation of metal nanoparticles in physiological solutions and in more stringent conditions where NaCl concentrations of 2 M are always tolerated and often 5 M NaCl is without effect on the stability of the nanoparticles.

b) Stable across a wide pH range (4-11).

c) Stable across a wide range of temperatures (below the freezing point of water to at least 120° C.).

d) No charge borne, neutral.

e) Absence of non-specific absorption to chromatography matrices typically used for the separation of biological molecules.

e) No non-specific binding to biological molecules, including in complex environments such as cell cultures in medium with serum.

f) No ligand exchange over 4 hours.

g) Easy to introduce specific functions through a functional PEG or functional peptide-ol or functional peptide.

h) Multiple functions can have different spatial relationships if they are on peptide-ol(s)/peptides and PEG and on peptide-ols/peptides with different sequences of amino acids.

In accordance with a further aspect of the present invention, there is also provided a method of producing a nanoparticle conjugate as described in any preceding claim by incubating in aqueous medium, a nanoparticle solution with a mixture of peptide-ols and/or PEG compounds.

The method may also include one or more ligands and optionally one or more identification means and/or functional groups which are conjugated to the peptide-ol and/or PEG compound prior to incubation with the nanoparticle or during the course of the incubation.

The method of producing a nanoparticle conjugate may additionally employ the use of freeze drying so that the nanoparticle conjugate can be stored or transported prior to use. It will be apparent that this may be required for certain ligands that may degrade or denature over time.

The method of producing a nanoparticle conjugate may additionally employ the use of boiling and/or autoclaving so that the nanoparticle conjugate may be sterilised prior to use, which may be required for certain applications.

In accordance with a further aspect of the present invention, there is provided a method of producing a nanoparticle conjugate as described hereinabove comprising the use of centrifugation

In accordance with a further aspect of the present invention, there is provided a method of producing a nanoparticle as claimed in any one of claims 1 to 34 comprising the steps of a) solubilisation of the nanoparticle in aqueous buffer, and b) centrifugation.

The nanoparticle conjugates made from light absorbing materials such as those containing noble metals and semi-conductors can be used as molecular interaction sensors. The “colour” of such nanoparticles depends on their size and for noble metal containing nanoparticles size may be changed by simply bringing two or more nanoparticles into close association (at the nm scale, so that it is representative of the protein scale) such that their dipoles couple. Nanoparticle conjugates incorporating identification means and/or functional groups can therefore be used as molecular interaction sensors, such as a receptor dimerisation sensor. Such sensors would be highly efficient (high sensitivity, no background, low amounts of macromolecules required) in high throughput screening applications in order to search for compounds whose activity is exerted by preventing or enhancing a molecular interaction. Such sensors would also allow highly efficient detection of a molecule(s) that causes dimerisation or oligomerisation of the “receptors”

The nanoparticle conjugates may be used for analysis of complex secondary gene products. For example, glycomics is an area which suffers from the fact that synthesis of glycans is not template driven. Therefore analytical tools and assays are only as good as purification methods and the sensitivity of detection systems. Nanoparticle conjugates with a saccharide binding function, e.g., hydrazide for reducing sugars and mercury adduct for sugars with an unsaturated bond, would increase sensitivity by several orders of magnitude. Users employing this method would be research laboratories utilising screening assays, etc.

The nanoparticle conjugates may also be used in bioelectronics applications, which so far have up until now been largely confined to using DNA as the scaffold. The interactions from any bioassay in can be used in bioelectronic device assembly. Moreover, many such interactions lend themselves to switching. One example would be coupling a nanoparticle to a redox group or protein, e.g., azurin, to form an actuator. Further examples may include phosphorylation-dephosphorylation and Ca²⁺-induced conformation changes and consequent binding reactions and phosphorylation-dephosphorylation reactions of the hydroxyl groups of the amino acids serine, threonine, tyrosine and the amino acid histidine. In some cases the organic material may be partially or completely removed, sometimes by means that fuse the nanoparticles to exploit the structures or linkages between the nanoparticles afforded by the ligands. In short, by virtue of the specificity and range of the tags, which may be placed on the peptide-ol/PEG shell, the range of combinatorial ordered assemblies available to bring together nanoparticles augments considerably the applications in bioelectronics.

The Applicants have assessed the folding of the pentapeptide-ol from the well-established principles of protein folding. CVVVT-ol, CSSSS-ol

CALNN-ol, CAVLT-ol, CAVYT-ol, CAVLY-ol, CAVVY-ol, CVLLY-ol, CVLIT-ol, CVDVT-ol, CVKVT-ol, CFFFT-ol were chosen as examples from the 6.4 million possible pentapeptide-ol sequences synthesized from the 20 amino acids found in proteins (20⁵*2, since sequences can be in two directions). CVT-ol, CVVT-ol were chosen as examples of shorter sequences, CVVVVTol, CCVVVVT-ol, CALVVVVT-ol as examples of longer sequences. The key features are:

At least one thiol, embodied in the above examples by cysteine at one end of the pentapeptide-ol to provide a strong affinity for gold.

A core sequence following the thiols)/cysteine(s) that will provide for good packing within the self-assembled monolayer of the matrix ligand shell. Features that provide for good packing are explained in the chapters on protein structure in standard undergraduate biochemistry textbooks, for example, “Lehninger Principles of Biochemistry” Chapter 2 (p 47) to Chapter 4 (page 156), by David L Nelson and Michael M Cox, Fourth Edition, W.H. Freeman, New York. In essence there are three features, the maximisation of inter- and/or intra peptide hydrogen bonding by the amide of the peptide bond and the concomitant reduction of hydrogen bonds between the peptide bond and the solvent, water, the maximal shielding of hydrophobic side chains from water and the preference of certain amino acids for particular conformations, such as alpha helical or beta strand. The exemplar sequences meet these criteria.

The present invention will now be more particularly described with reference to the following example and figures.

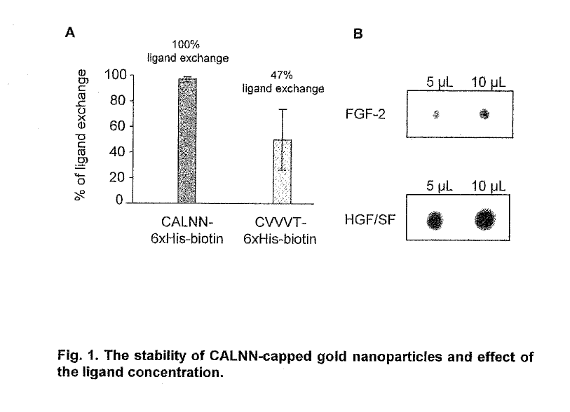

FIGS. 1 A, B is a graph of the results of an experiment conducted to show the lack of biological stability of a nanoparticle having a peptide shell comprising peptide having the sequences of CALNN;

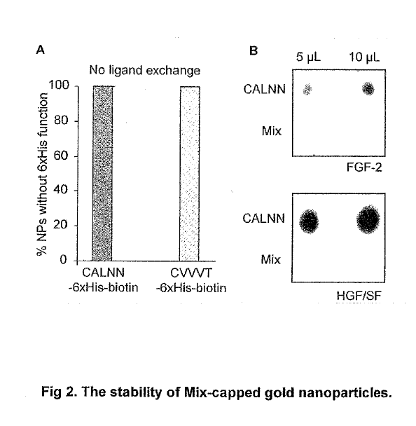

FIGS. 2 A, B is a graph of the results of an experiment conducted to show the biological stability of a nanoparticle having a shell comprising peptide-ol (of the sequence CVVVT-ol and thiolated alkane PEG;

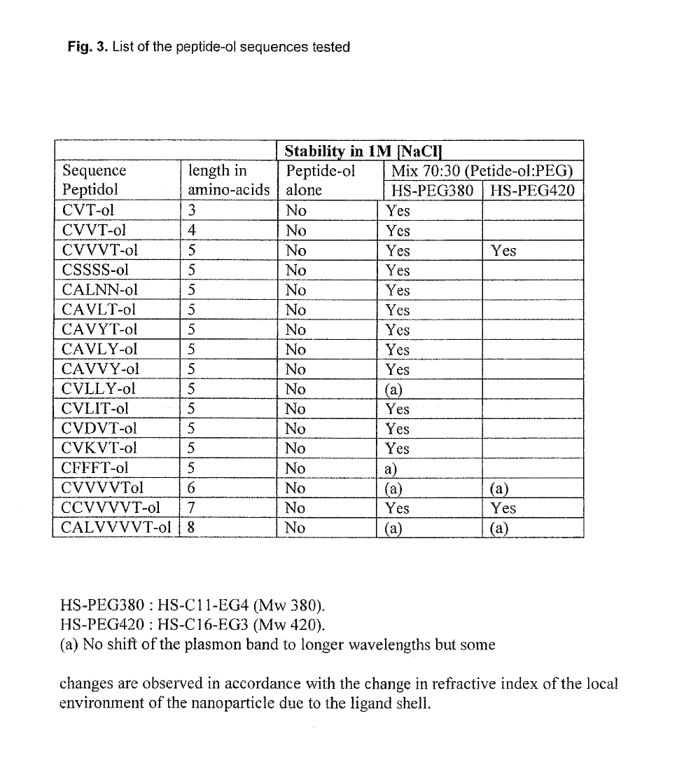

FIG. 3 is a table listing results of an experiment to test different peptide-ol sequences for their ability to stabilise gold nanoparticles against electrolyte-induced aggregation along and in combination with PEG compounds.

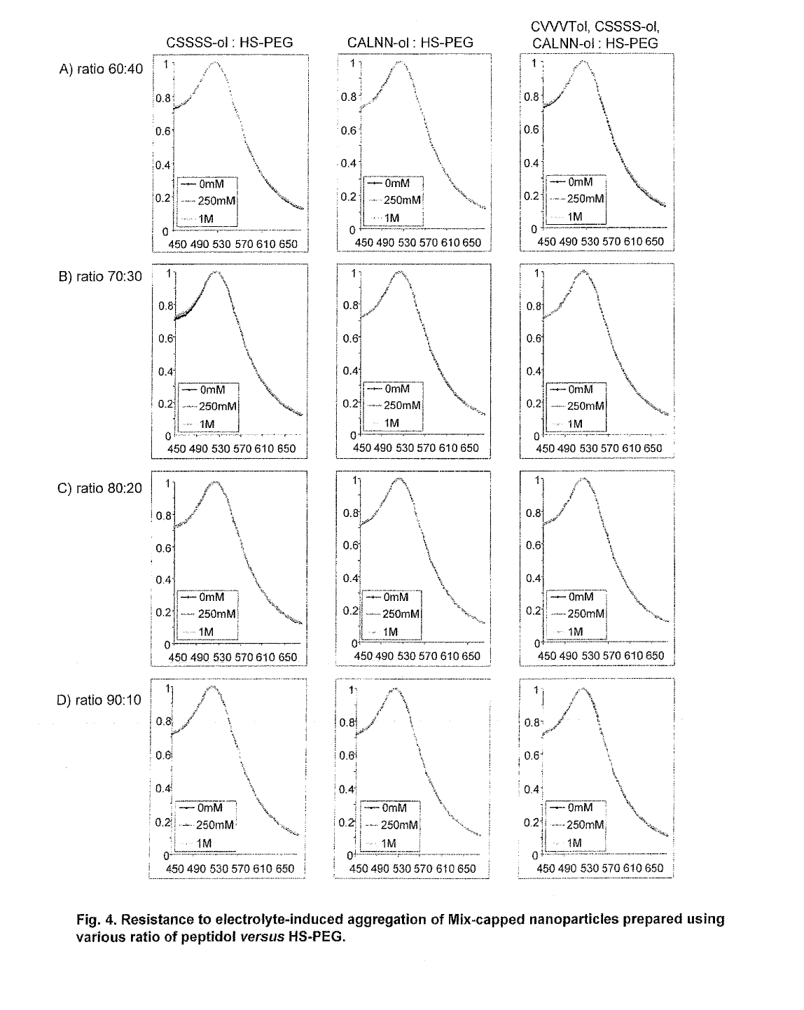

FIGS. 4. A-D are graphs of the results of an experiment conducted to show the salt stability of compositions according to the present invention;

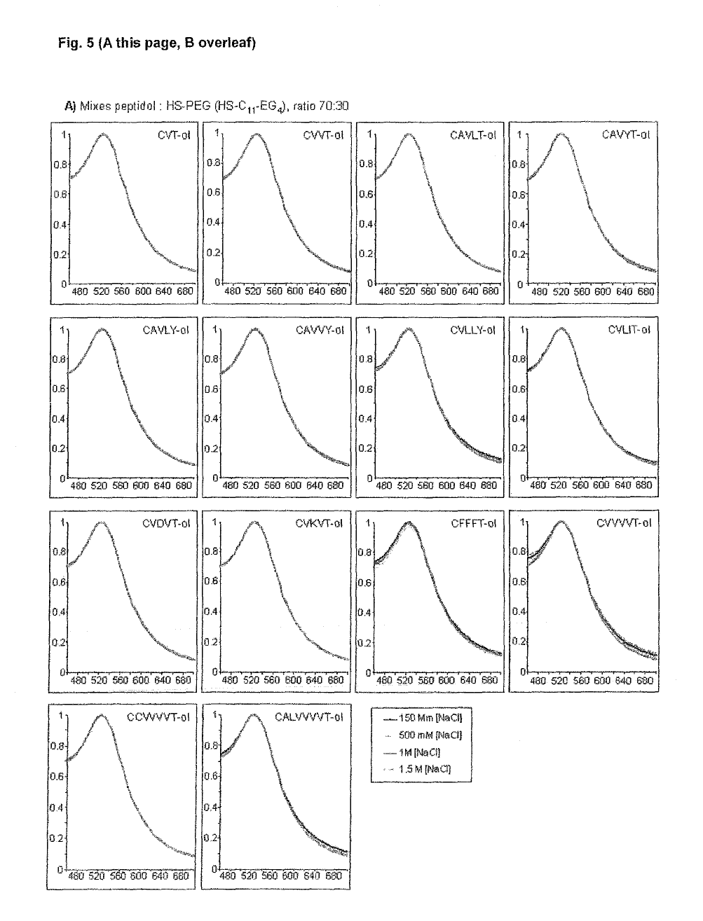

FIGS. 5 A, B are graphs of results of an experiment conducted to show the salt stability of compositions according to the present invention

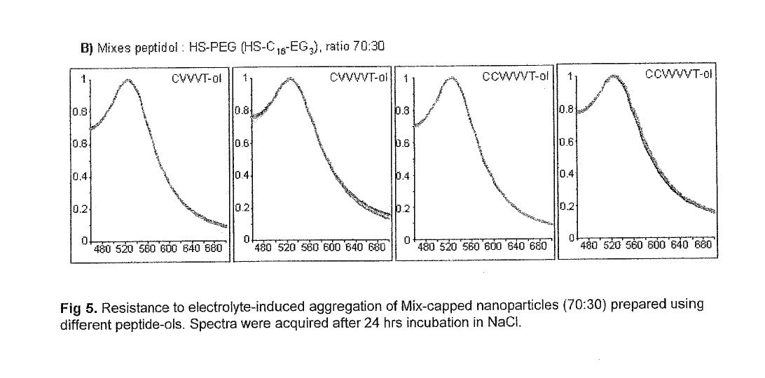

FIG. 6 is a graph of the results of an experiment conducted to show the stability of autoclaved compositions according to the present invention;

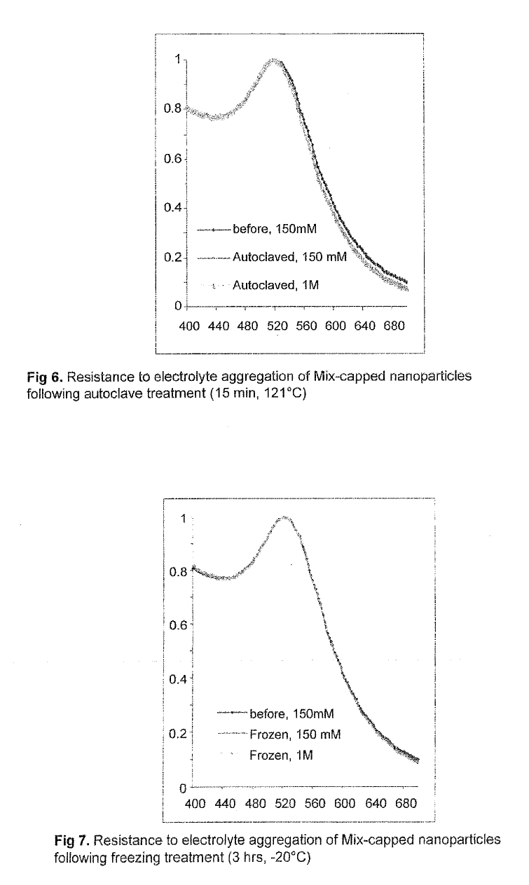

FIG. 7 is a graph of the results of an experiment conducted to show the effect of freezing compositions according to the present invention and the stability thereof;

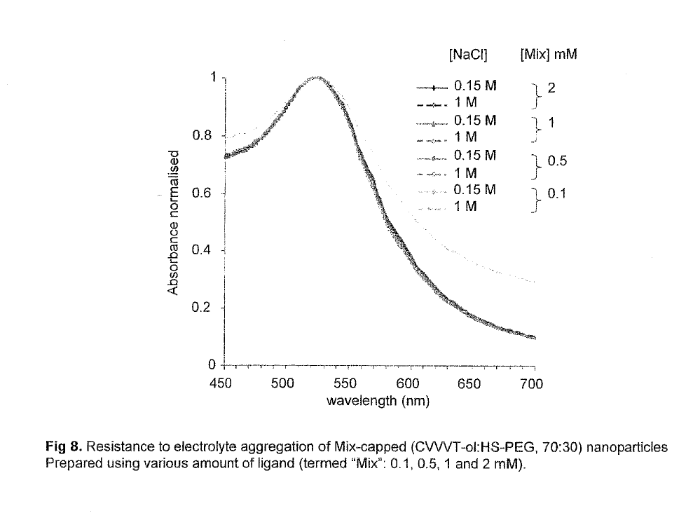

FIG. 8 is a graph of the results of an experiment conducted to show the results of an experiment conducted to show the dependence of the stability of a preferred nanoparticle on the ligand concentration;

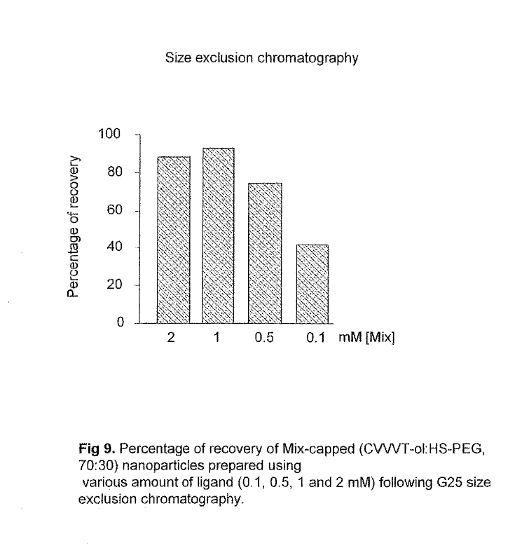

FIG. 9 is a histogram of the results of an experiment conducted to show the dependence of the recovery of nanoparticles on the concentration of ligand according to the present invention from a Sephadex G25 size exclusion column;

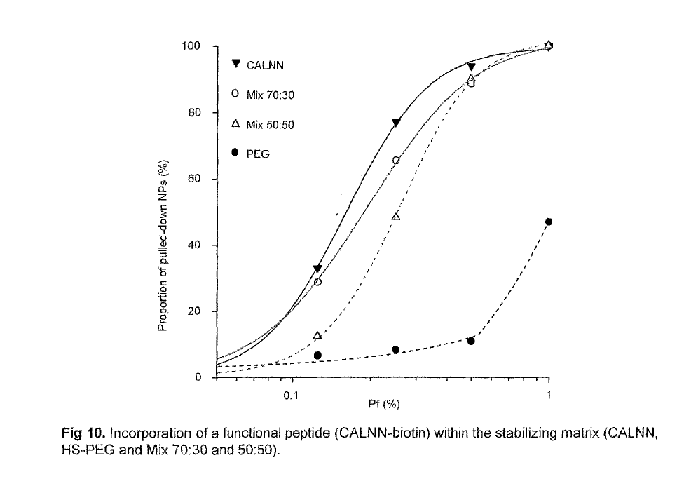

FIG. 10 is a graph of the results of an experiment conducted to show matrix ligand mix facilitates incorporation of peptide-ols with a function;

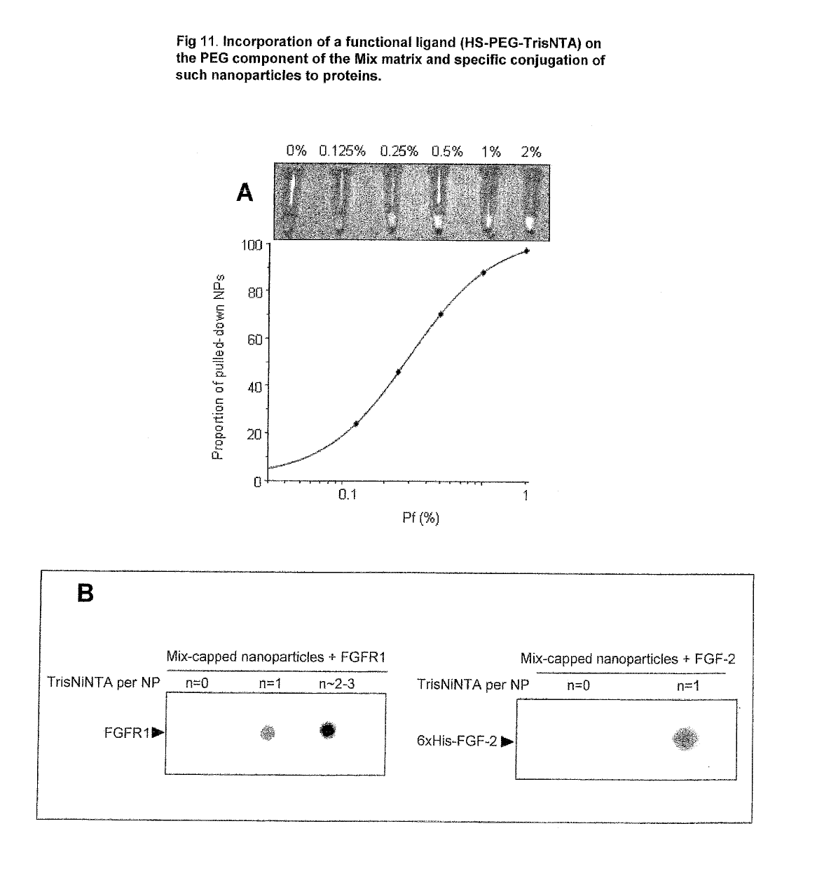

FIGS. 11A, B are the results of an experiment conducted to show functionalisation of the matrix ligand mix with a thiolated PEG incorporating a TrisNTA function and the specific conjugation of these nanoparticles to the protein FGFR1;

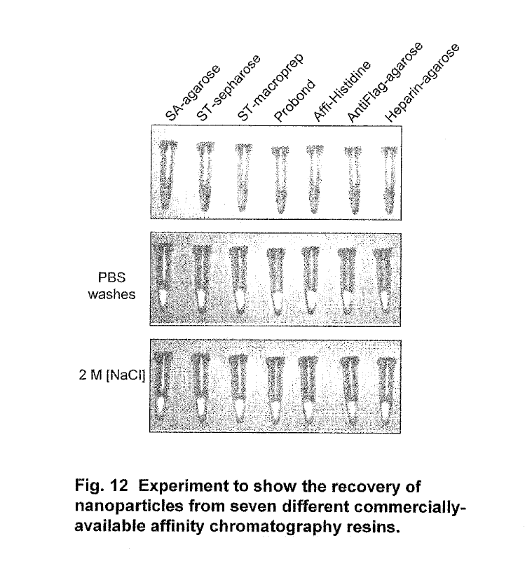

FIG. 12 are the results of an experiment conducted to show the recovery of nanoparticles according to the present invention from seven different common affinity chromatography resins;

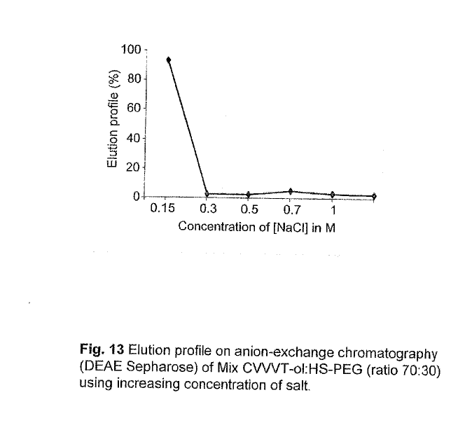

FIG. 13 are the results of an experiment conducted to show the recovery of the nanoparticles prepared with different concentration of ligand mix from a Sepharose DEAE anion-exchange column;

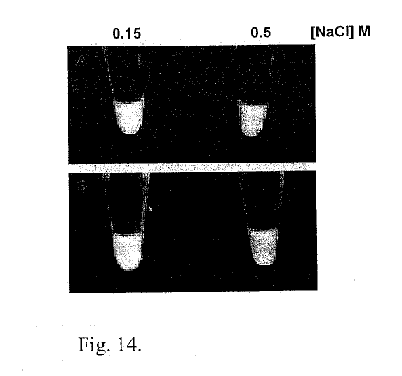

FIG. 14 shows the results of an experiment in which Q-dots in accordance with the present invention were prepared using the THF method and the Mix matrix (Mix 50:50 (v/v) HS-PEG:CVVVT-ol) as ligand;

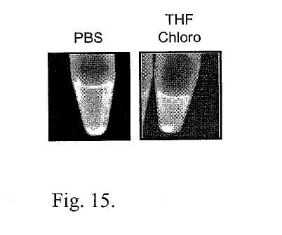

FIG. 15 shows the results of an experiment in which Q-dots in accordance with the present invention were prepared using the PBS and the THF/Chloroform method;

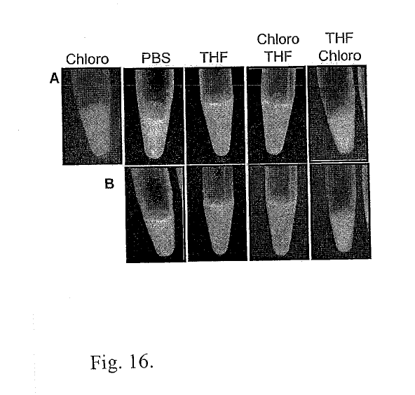

FIG. 16 shows the results of an experiment in which Q-dots in accordance with the present invention were prepared using all 5 methods and HS-PEG as matrix ligand;

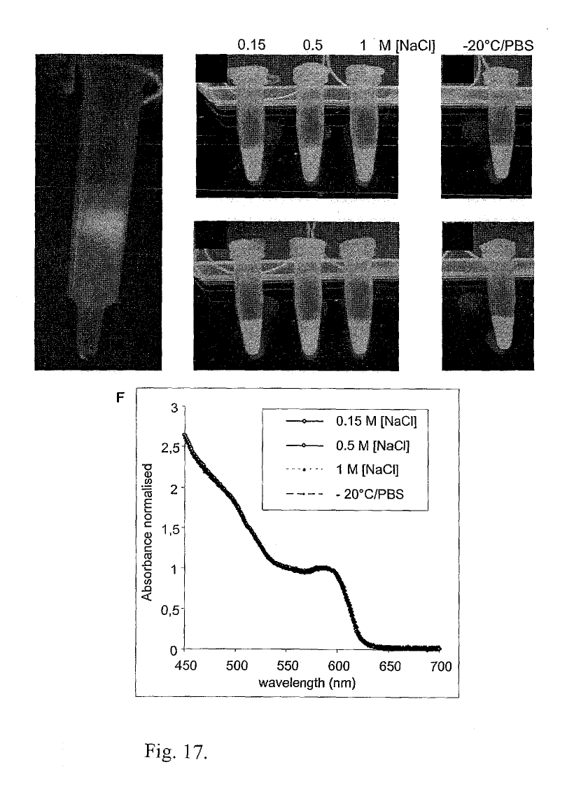

FIG. 17 shows the results of an experiment in which Q-dots in accordance with the present invention were prepared using the THF method and HS-PEG as matrix ligands; and

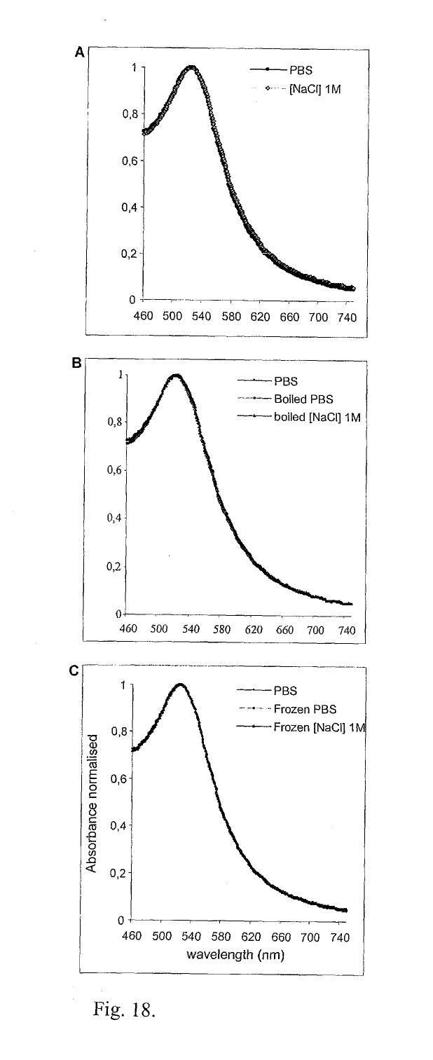

FIG. 18 shows the Stability against electrolyte-induced aggregation of PEPEG-capped gold nanoparticles following boiling or freezing treatments.

During experiments, a peptide-ol (typically a pentapeptide-ol with one or more cysteine residues, which contain a thiol at the N-terminus, and an alcohol group in place of the natural carboxyl group at the C-terminus (hence termed peptide-ol)), a thiolated alkane PEG and a peptide ethylene glycol were investigated for use in stabilising nanoparticles. Sequences that have been found to provide stability in the mixed matrix shell system are:

CVT-ol, CVVT-ol, CVVVT-ol, CSSSS-ol CALNN-ol, CAVLT-ol, CAVYT-ol, CAVLY-ol, CAVVY-ol, CVLLY-ol, CVLIT-ol, CVDVT-ol, CVKVT-ol, CFFFT-ol, CVVVVTol, CCVVVVT-ol, CALVVVVT-ol

In mixtures with thiolated alkyl PEG, all seventeen peptide-ol sequences provide for stabilisation, and CVVVT-ol was one of the most effective.

The second component for the nanoparticle conjugate was the thiolated alkyl PEG. Typically this has a thiol group attached to a C11 alkyl chain with a pendent PEG unit. The length of the alkyl chain wash matched to that of the peptide-ol though different lengths of alkyl chains can be accommodated. As few as two ethylene glycol units were found to be required for stabilisation, typically 3-4 ethylene glycol units are used.

The third component for the nanoparticle conjugate was the peptide ethylene glycol. The sequence considerations for the peptide moiety are similar to those for the peptidol and CVVVT(EG)₄ol was used as an exemplar.

Nanoparticles were found to be stable with up to 90:10 (mole/mole) peptide-ol:PEG, though the highest stability nanoparticles require at least 70:30 (mole/mole) peptide-ol/PEG. At low peptide-ol levels (down to 100% thiolated alkyl PEG), the performance of the matrix shell is compromised in an important way. At low peptide-ol %, the incorporation of peptide-ols and peptides with a functional group is severely compromised (FIG. 10). Thus, at intermediate ratios of peptide-ol and thiolated alkyl PEG (typically 30:70 to 80:20), stability of the nanoparticles is optimal as is the incorporation of peptide-ols with a function(s). Functions can also be incorporated in the thiolated alkyl PEG (FIG. 11), though the ease of peptide-ol and peptide synthesis means that functions will most often be incorporated in the peptide-ol.

Surprisingly, given the weaker thiol bond, quantum dots were found to be stabilised by both alkane thiol PEG alone and by the mix peptidol/alkanethiol PEG. Since the quantum dots are prepared in organic solvent with ligands that present hydrophobic entities to solvent, ligand-exchange was necessary. Five different ligand exchange procedures were tested, the key being to reduce the concentration of free hydrophobic ligand in solution prior to the exchange process. Key second step was to take the water soluble quantum dots and reload them with the ligands affording solubility in water and stability in biological environments, the peptidol, peptide ethylene glycol and/or the alkane thiol PEG.

### EXAMPLES

An experiment was conducted to produce a nanoparticle conjugate in accordance with the present invention.

Materials: The CVVVT-ol (T-ol being for Threoninol) was purchased from Anaspec (San Jose, US) and Polyethylene glycol (HS-PEG, HSC11EG) was purchased from ProChimia (ProChimia Surfaces, Poland). The 10 nm gold nanoparticles (G-NPs) were purchased from British Biocell (BBInternational Ltd, UK). Sephadex G25 superfine and Tween 20 were purchased from Sigma-Aldrich Ltd (Dorset, UK).

Preparation of Mixed Matrix-Capped Gold Nanoparticles:

In the following, PBS is defined as Phosphate-Buffered Saline (8.1 mM Na2NP04, 1.2 mM KH2P04, 150 mM NaCl and 2.7 mM KCl, pH 7.4) and 10×PBS is a 10 times more concentrated solution of the same salts. The peptide-ol stock solutions was prepared by dissolving the peptide-ol powder in DMSO/milliQ H2O (25:75, v/v) at 4 mM final concentration. This stock solution was then aliquoted and kept at −20° C. For the HS-PEG (HSC11EG) molecule, a stock at 5 mM final concentration in methanol was prepared, aliquoted and stocked at −20° C. Before use, HS-PEG and CVVVT-ol molecules were diluted each at 2 mM final concentration using milliQ water and mixed together at a 30:70(v/v) ratio (or other as indicated in the figures). Gold nanoparticle solution was added to this mixed matrix ligand solution in a 10 to 1 volume ratio and 10×PBS were added to give a final 1×PBS concentration. The reaction was left overnight at room temperature on a wheel and the excess matrix ligand was then removed by size-exclusion chromatography using Sephadex G25 superfine as separation medium and PBS supplemented with Tween 0.005% (v/v) as the mobile phase.

FIG. 1 is a comparative example and clearly shows that nanoparticles having a shell made up of the peptide CALNN does not possess good biological stability. CALNN-capped gold nanoparticles were prepared using starting concentration of 2 Mm of CALNN peptide. Following purification, the stability of the nanoparticles was tested with respect to ligand exchange and a specific binding of proteins. (A) Ligand exchange experiment: CALNN-capped nanoparticles were incubated with CVVVT-6×His-Biotin and CALNN-6×His-biotin peptides and purified. The percentage (%) of ligand exchange correspond to the percentage of nanoparticles having incorporated at least one 6×His-biotin functional peptide within the matrix so pulled down by nickel chelating resin (B) A specific binding of proteins: CALNN-capped nanoparticles were incubated with FGF-2 or HGF/SF proteins and purified. Five and 10 μL of nanoparticles at 10 nM concentration were dotted onto PVDF membrane and protein were detected by Dot-Blot. These nanoparticles are susceptible to ligand exchange over 4 hours (FIG. 1A; x-axis time hours, y-axis, percent of nanoparticles having exchanged at least one ligand) and they bind proteins a specifically, as shown by the dot blot (FIG. 1B) where the nanoparticles are shown to have bound the proteins FGF-2 and HGF/SF.

In contrast, the nanoparticles having a shell made up of a peptide-ol (CCVVVT-ol) and PEG compound are completely resistant to such ligand exchange (FIG. 2 A) and do not bind proteins non-specifically (FIG. 2 B, “CALNN” denotes the same nanoparticles as in FIG. 1 for comparison, “mix” denotes nanoparticles prepared according to the present invention). The term “mix” refers to the peptidol-alkane thiol PEG matrix mixture. If the ratio is not specified in a figure then is it 70% peptidol to 30% alkane thiol PEG. If the sequence of the peptidol is not specified, then it is CVVVT(ol). In FIG. 2, (A) Ligand exchange experiment: Mix-capped nanoparticles (CVVVT-ol:HS-PEG, ratio 70:30) were incubated with CVVVT-6×His-Biotin and CALNN-6×His-biotin peptides and purified. The percentage (%) of NPs without 6×His function correspond to the percentage of nanoparticles that didn't incorporate an 6×His-biotin functional peptide within the matrix so not pulled down by nickel chelating resin (B) A specific binding of proteins: Mix-capped nanoparticles (CVVVT-ol:HS-PEG, ratio 70:30) were incubated with FGF-2 or HGF/SF proteins and purified. Five and 10 μL of nanoparticles at 10 nM concentration were dotted onto PVDF membrane and protein were detected by Dot-Blot. Results obtain with CALNN-capped nanoparticles during the same experiment is shown as comparison point.

In FIG. 3 seventeen different peptide-ol sequences are shown, which all stabilise nanoparticles against electrolyte-induced aggregation when used in accordance with the present invention, whereas alone they to not stabilise the nanoparticles.

In FIG. 4 a series of UV-visible absorption spectra. (uv-vis spectra) showing the stability of nanoparticles synthesised with three different peptide-ols at various ratios of peptide-ol:PEG. Spectra were acquired after 24 hrs incubation in sodium phosphate buffer pH 7.4 supplemented with 0, 250 mM or 1M NaCl. Aggregation results in a rightwards shift of the spectrum; in the extreme the plasmon band at 518-530 disappears to be replaced by one ≧600 nm). The overlap of the spectra shows clearly that increasing the concentration of electrolytes (0 M-1M NaCl) has no effect on the stability of the nanoparticles and that different ratios of the peptide-ols CSSSS-ol, CALNN-ol and of a mix of three peptide-ols (CVVVT-ol, CSSSS-ol, CALNN-ol; ratio is for total peptide-ol) and HS-PEG are equally stable.

In FIG. 5A a series of UV-visible absorption spectra. (uv-vis spectra) showing the stability of nanoparticles synthesised with 14 different peptidols at a ratio of 70:30 peptide-ol:PEG, where the thiolated PEG has a C11 alkane chain and four ethylene glycol units (EG4). The overlap of the spectra shows clearly that the concentration of electrolytes (0-1.5 M NaCl has no effect on the stability of the nanoparticles.

In FIG. 5B a series of UV-visible absorption spectra. (uv-vis spectra) showing the stability of nanoparticles synthesised with 4 different peptidols at a ratio of 70:30 peptide-ol:PEG where the thiolated PEG has a C16 alkane chain and three ethylene glycol units (EG3). The overlap of the spectra shows clearly that the concentration of electrolytes (0-1.5 M NaCl has no effect on the stability of the nanoparticles.

FIG. 6 shows the plasmon absorption peak of autoclaved nanoparticles Autoclave. Nanoparticles synthesized with 2 mM matrix ligand matrix mix may be autoclaving of nanoparticles for 15 min 121° C. (a standard sterilisation protocol) retain their stability with respect to electrolyte-induced aggregation; the plasmon absorption peak of the autoclaved nanoparticles is not affected by NaCl concentrations of at least 1 M.

FIG. 7 shows effect of Freezing on the stability of nanoparticles according to the present invention. Nanoparticles synthesized with 2 mM matrix ligand mix may be frozen. Nanoparticles were frozen at −20° C. min for 4 hours and their stability with respect to electrolyte-induced aggregation was measured; the plasmon absorption peak of the frozen nanoparticles is not affected by NaCl concentrations of at least 1 M.

FIG. 8 This shows the preferred embodiment of the method: matrix ligand mix concentration must be 0.5 mM or above for the synthesis of nanoparticles that are resistant to electrolyte-induced aggregation.

FIG. 9 shows the recovery of nanoparticles from a Sephadex G25 size exclusion column. The preferred embodiment for the synthesis of nanoparticles that are to be efficiently subjected to size exclusion chromatography is 1 mM matrix ligand mix; at 0.5 mM matrix ligand mix there is a small loss of material on the column, which is severe at 0.1 mM matrix ligand mix.

FIG. 10 shows that the matrix ligand mix facilitates incorporation of peptides with a function. Capped nanoparticles were prepared with different proportion of functional peptide (Pf). The percentage of biotinylated nanoparticles is measured by the proportion of nanoparticles pulled-down by Streptavidin-agarose beads. The functional peptide CALNNGKGALVPRGSGK(biotin)TAK (termed CALNN-biotin in the figure) is efficiently incorporated into nanoparticles with the same pentapeptide (CALNN, a standard peptide for comparison to previous work, Levy et al., (2006) A generic approach to monofunctionalized protein-like gold nanoparticles based on immobilized metal ion affinity chromatography. ChemBioChem 7:592-594). This functional peptide, (CALNN-biotin) is similarly incorporated into the matrix ligand shell of 70:30 peptidol(CVVVT-ol):PEG (C11, EG4) matrix ligand mix nanoparticles. It is incorporated less efficiently into 50:50 peptide-ol-PEG matrix ligand mix nanoparticles (a higher mole percentage of functional peptide must be added to the matrix ligand mix to obtain a similar level of incorporation). It is not incorporated effectively into a 100% alkane thiol PEG matrix ligand shell, due to the adverse environment of the CALNN sequence (surrounded entirely by alkyl chains). This will reduce the statistical control (Levy et al., op. cit.) over the number of functional peptide-ols incorporated into the nanoparticles.

FIG. 11 shows that the matrix ligand mix facilitates incorporation of PEG compounds with a function. FIG. 11 (A) Capped nanoparticles (CVVVT-ol:HS-PEG, 70:30) were prepared with different proportions of HS-PEG-TrisNTA (Pf). The percentage of functionalized nanoparticles is measured as the proportion of to nanoparticles pulled-down by Affi-His beads. The photograph of the tubes above the graph shows the increasing amount of nanoaparticles pulled down with the pellet of Affi-His. (B) Specific and stoichiometric attachment of TrisNiNTA Mix-capped nanoparticles to the FGFR1 and the FGF2 proteins. Mix-capped nanoparticles without TrisNiNTA do not bind FGFR1 or FGF2 a specifically. Nanoparticles with a single TrisNiNTA (n=1) are specifically conjugated to FGFR1 and FGF2, as seen by the immunoreactivity in the dot-blot. When more TrisNiNTA are incorporated into the nanoparticle ligand shell (n−˜2-3), the number of proteins conjugated per nanoparticle increases, as does the immunoreactivity. Equal amounts of nanoparticles were loaded onto each spot of the dot blot. The functional PEG HS-C11-EG4-TrisNiNTA is efficiently incorporated into the nanoparticles, with good control over the valency of functionalisation (FIG. 11A). The functionalized nanoparticles can then be conjugated through the TrisNiNTA function to a protein, in this instance FGFR1, which has a hexahistidine tag at its N-terminus ((Duchesne et al. (2006) N-glycosylation of fibroblast growth factor receptor 1 regulates ligand and heparan sulfate co-receptor binding. J. Biol. Chem. 281: 27178-27189) and FGF2, similar to that described (Duchesne et al. op. cit.), but with a 6× histidine tag at its N-terminus.

FIG. 12 shows the recovery of nanoparticles from seven different commercially-available affinity chromatography resins. Nanoparticles are in the supernatant, not the chromatography gel pellets, which is further evidenced by these pellets being clear following washes with PBS and 2 M NaCl. The peptidol:PEG nanoparticles do not bind a specifically to any of these chromatography resins, since the nanoparticles remain in the solution, rather than concentrating in the chromatography resin, which has settled at the bottom of the tube. Moreover, after washes with PBS and 2 M NaCl, no nanoparticles have remained associated with the chromatography resin pellet, as this is not coloured. SA-agarose is streptavidin agarose, ST-Sepharose is streptactin Sepharose, ST-macroprep is streptactin macroprep, Probond is an immobilised metal affinity chromatography resin, Affi-Histidine is Affi 10 Gel functionalised with a peptide containing a hexahistidine tag, AntiFlag agarose has an immobilised antibody to the “Flag tag” a functionalisation sequence in common use, heparin agarose is an agarose functionalised with the polysaccharide heparin.

FIG. 13 shows the recovery of nanoparticles from a Sepharose DEAE anion-exchange column. The nanoparticles do not bind to the column, since they elute with the PBS (0.15 M naCl) load. The peptidol:PEG nanoparticles have no charge and do not bind to the DEAF. Anion-exchange would be particularly pertinent with such nanoparticles functionalised with anionic entities, e.g., nucleic acids, anionic polysaccharides, since they can then be separated on the basis of the structure of the conjugated entity, without interference from the nanoparticle probe.

### Preparation of Ligand-Capped Q-Dots

Six different experiments were performed to define satisfactory conditions for the replacement of the hexadecylamine ligand shell on the Q-Dots, which imparts stability in organic solvents, but not in aqueous solutions with ligands suitable for biological applications, PEG (Hs-C₁₁-EG₄) or Mix matrix (HS-PEG:CVVVT-ol) ligands. The term “matrix ligand” refers to either PEG or the Mix. Q-Dots used were from Sigma (Lumidot, 4 μM in toluene, λₑₘ 610 nm). All reactions were performed in the dark.

The five experiments have a distinct first step (Step 1) and a common second step (Step 2). Step 1 allows the transition of the Q-Dots from organic solvents to aqueous buffers. Step 2 reloads ligand into the ligand shell to impart maximum stability to the bio Q-Dots for biological applications. Since the ligands are identical to those used for gold nanoparticles, functionalisation would be accomplished in exactly the same way.

a) Step 1. Transition from Organic Solvent to Aqueous Phase

Chloroform method. 100 μL of Chloroform with 2 mM final concentration of matrix ligand (HS-PEG or Mix 50:50 (v/v) Hs-PEG:CVVVT-ol) were prepared and 2 μL of Q-Dots at 4 μM was added and mixed to this solution. The reaction was left incubated 90 min in the dark on a wheel and an equal volume of PBS containing 0.2 mM of matrix ligand (PBS-0.2 mM ligand) was added to the reaction. Following strong agitation, a centrifugation (5 minutes, 10000 rpm, RT) was performed to separate the organic from the aqueous phase. The aqueous phase containing the Q-Dots with HS-PEG or Mix Matrix was picked up and put in a separate tube. If needed, 1 volume of PBS-0.2 mM ligand (HS-PEG or Mix Matrix) was added again to the chloroform phase until all Q-Dots were extracted toward the aqueous phase. The Q-Dot solution was then centrifuged 5 min at 10000 rpm and the pellet (Q-Dot) resuspended in H₂0-T0.01% (H20-Tween-20). Excess ligands (especially the hexadecylamine, which was present in large excess in the initial solution of lumidot in toluene and which may still contaminate the sample), was removed by G-25 size-exclusion chromatography using H₂0-T as mobile phase.

PBS method: 100 μL of PBS with 2 mM final concentration of matrix ligand was prepared and 2 μL of Q-Dots at 4 μM were deposited on the top of this solution. Without prior agitation the solution was centrifuged for 30 min at 10 000 rpm at RT. The supernatant was removed and 100 μL of PBS containing 1 mM of ligand added to the pellet (Q-Dot). The solution was left to react overnight at 4° C. in the dark and then centrifuged 7 min at 10 000 rpm. The supernatant (Q-Dots in solution) was picked up and kept. However, some Q-Dots still remain in the pellet. Therefore, PBS-T0.01% (PBS-T) containing 0.1 to 0.2 mM ligand (PBS-T-ligand) was added to the pellet, the solution was vortexed, centrifuged and the supernatant containing the newly solubilised Q-Dots picked up. This centrifugation/resuspension step was repeated (usually 3 to 4 times) until all Q-Dots are well solubilised in the PBS-T-ligand solution. All soluble fractions were then put together, concentrated using Nanosep centrifugal ultrafiltration devices and the Q-Dots purified by G-25 size exclusion chromatography using H₂0-T as mobile phase.

THF method: Two μL of Q-dots at 4 μM was resuspended in 100 μL of Tetrahydrofuran (THF) solvent and one volume of PBS containing 1 mM of ligand was added and mixed to this solution. The reaction was left to react overnight at 4° C. (or 2 h RT). Q-dots were pelleted by centrifugation (5 min, 10 000 rpm) and resuspended in H₂0-T containing matrix ligands at 0.1 to 0.2 mM concentration. The solution was vortexed, centrifuged and the supernatant containing the solubilised Q-Dots picked up. However some Q-Dots still remained in the pellet. Therefore, this centrifugation/resuspension step was repeated (usually 2 to 3 times) using H₂0-T-ligand or PBS-T-ligand until all Q-Dots were all well solubilised. All soluble fractions were then pooled, concentrated using Nanosep centrifugal ultrafiltration devices and the Q-Dots purified by G-25 size-exclusion chromatography using H₂0-T as mobile phase.

THF/Chloroform method: Two μL of Q-dots at 4 μM was resuspended in 100 μL of Tetra Hydro Furan (THF) solvent and one volume of PBS containing 1 mM of ligand was added and mixed to this solution. The reaction was left reacted overnight at 4° C. (or 2 hrs RT). An equal volume of chloroform was then added and following strong agitation, a centrifugation (5 min, 10 000 rpm, RT) was performed to separate the organic from the aqueous phase. The aqueous phase containing the bio-functionalized Q-Dots was picked up, put in a separate tube and subjected to another chloroform extraction.

Following both extractions, the aqueous phase containing the Q-Dots is centrifuged. The supernatant (some of the Q-Dots solubilised) was kept and the pellet (remaining unsolubilised Q-Dots) resuspended with H₂0-T0.01% containing matrix ligands at 0.1 to 0.2 mM concentration. The solution was vortexed, centrifuged and the supernatant containing the solubilised Q-Dots picked up again. This centrifugation/resuspension step was repeated (usually 2 to 3 times) until all Q-Dots were well solubilised. All soluble fractions were then put together, concentrated using Nanosep centrifugal ultrafiltration devices and the Q-Dots purified by G-25 size exclusion chromatography using H₂0-T as mobile phase.

Chloroform/THF method: 100 μL of Chloroform with 1 mM final concentration of matrix ligand were prepared and 2 μL of Q-Dots at 4 μM was added and mixed to this solution. The reaction was left to incubate 90 min in the dark on a rotating wheel and an equal volume of PBS containing 0.2 mM of matrix ligand (PBS-0.2 mM ligand) was added to the reaction. Following strong agitation, a centrifugation was performed to separate the organic from the aqueous phase. The aqueous phase containing the bio-functionalized Q-Dots was picked up and put in a separate tube. If needed, 1 volume of PBS-0.2 mM ligand was added again to the chloroform phase until all Q-Dots were extracted toward the aqueous phase.

An equal volume of THF containing 1 mM of ligand was then added to the aqueous fraction containing the Q-Dots and the reaction is left incubated 2 hrs at RT on a wheel. Remaining protocol is as described for the THF method.

### b) Step 2. Optimisation of the Matrix at the Surface of the O-Dot

All methods cited above allow solubilisation of the Q-Dots in aqueous buffer (H20, PBS). However, following this first step it is most probable that the self-assembled matrix protecting the Q-Dot is still not highly compacted and so is not suitable for biological application, since the ligand concentration attainable in the first step is low due to the solvent mixture and the presence of the outgoing hexadecylamine ligand. By analogy with gold nanoparticles, the product of the first step resembles most the product of synthesis of gold nanoparticles at lower, so suboptimal, concentrations of ligand, e.g., less than 0.5 mM ligand in FIG. 8 and less than 1 mM ligand in FIG. 9. Following the G-25 chromatography or ultrafiltration, the Q-Dot solution is concentrated using a Nanosep centrifugal ultrafiltration devices (cut-olf 10 kDa) and resuspended in 200 μL of PBS containing 0.2 mM of matrix ligand to reload the Q-dot ligand shell. The reaction is left reacted overnight or more and, when needed, excess ligand is removed using Nanosep centrifugal ultrafiltration devices or G-25 size-exclusion chromatography. FIG. 14 shows the results of an experiment in which Q-dots were prepared using the THF method and the Mix matrix (Mix 50:50 (v/v) Hs-PEG:CVVVT-ol) as ligand. Five M NaCl were added to an aliquot to obtain a final concentration of 0.5 M NaCl. Another aliquot was mock treated by addition of PBS. Samples were vortex and left over night (14 h) at 4° C. before picture acquisition. Panel A shows the fluorescence in PBS (0.15 M NaCl) and the 0.5 M NaCl solution (white colour), indicating the quantum dots are stable are stable (still fluorescent) and not in large aggregates (fluorescence is in solution). Panel B shows that after centrifuging these same samples at 10000 g for 5 min the fluorescence remained in solution and so not microaggregated, but truly dispersed.

FIG. 15 shows the results of an experiment in which Q-dots were prepared using the PBS and the THF/Chloro method and the Mix matrix (Mix 50:50 (v/v) HS-PEG:CVVVT-ol) as ligand. Pictures of the Q-Dots in solution in PBS were acquired following a 5 min centrifugation at 10000 rpm to detect the presence of eventual aggregates.

FIG. 16 shows the results of an experiment in which Q-dots were prepared using all 5 methods and HS-PEG as matrix ligand. (A) Picture of the Q-Dots in solution in PBS. (B) Samples were then centrifuged at 10000 g for 5 min to detect the presence of eventual aggregates.

FIG. 17 shows the results of an experiment in which Q-dots were prepared using the THF method and Hs-PEG as matrix ligands. (A) Picture of the size exclusion chromatography column used to remove the excess ligands between Step 1 and the Step 2.

(B,C) Five M NaCl were added to an aliquot of the PBS solubilised Q-Dots to obtain a final concentration of 0.5 or 1M NaCl. Another aliquot was mock treated by addition of PBS. Samples were vortex and left over night (18 h) at 4° C. before picture acquisition (B). Samples were then centrifuged at 10000 rpm for 5 min to detect the presence of eventual aggregates (C).

(D,E) An aliquots of Q-Dots in PBS was frozen (overnight) at −20° C. and defreezed before picture acquisition (D). The sample was then centrifuged at 10000 rpm for 5 min to detect the presence of eventual aggregates (E).

(F) Absorbance spectrum (normalised) of the samples shown in (B) (after 18 hrs incubation in salt).

(A) to (F) were performed using the same preparation of PEG-capped Q-Dots.

### Preparation of Peptide Ethylene Glycol-Capped Gold Nanoparticles.

CVVVT-EG₄-ol (EG being for ethylene glycol) was purchased from Cambridge Research Biochemicals (Cleveland, UK). The 10 nm gold nanoparticles (G-NPs) were purchased from British Biocell (BBInternational Ltd, UK).

In the following example PBS is to be understood to mean Phosphate-Buffered Saline (8.1 mM Na₂HPO4, 1.2 mM KH₂PO4, 150 mM NaCl and 2.7 mM KCl, pH 7.4) and 10×PBS for 10 times more concentrated solution of the same salts. The CVVVT-EG₄-ol ligand, (peptide-ethylene glycol, referred as PEPEG) was resuspended at 10 mM final concentration using DMSO, aliquoted and kept at −20° C. Before used, the PEPEG ligand was diluted at 2 mM using milliQ H₂0. Gold nanoparticles solution was then added to this ligand solution in a 10 to 1 volume ratio and 10×PBS was added for a 1× final concentration. The reaction was left overnight at room temperature on a wheel and the excess ligand was removed using Nanosep centrifugal ultrafiltration devices.

UV-Visible Spectrometry: absorption spectra were recorded at room temperature using a Spectra Max Plus spectrophotometer (Molecular Devices, Wokingham, U.K.).

Stability of PEPEG capped nanoparticles: PEPEG-capped nanoparticles were prepared and stored for 3 months at 4° C. before use, which shows their long-term stability. The capped nanoparticle sample was split into 3 tubes. One was left at 4° C., one boiled 10 minutes at 100° C., left 30 min at room temperature and then frozen at −4° C. for 72 h. The second tube of PEPEG nanoparticles was frozen 72 h at −20° C., whereas the third tube was simply left at 4° C. Following these treatments the contents of all 3 tubes were split into two and NaCl 5M was added to one tube to obtain a final concentration of 1M NaCl. The second tube was mock treated using PBS. Absorption spectra were recorded at room temperature using a Spectra Max Plus spectrophotometer (Molecular Devices, Wokingham, U.K.).

FIG. 18 shows the stability against electrolyte-induced aggregation of PEPEG-capped nanoparticles following boiling or freezing treatments. PEPEG-capped gold nanoparticles (10 nm) were prepared and stored for 3 months at 4° C. prior to the stability test. (A) electrolyte-induced aggregation was determined by measuring the absorbance spectra after 8 h incubation in sodium phosphate buffer 10 mM, pH 7.4 supplemented with 150 mM (PBS) or 1 M of NaCl. (B) PEPEG-capped nanoparticles in PBS were boiled for 10 min at 100° C. and then kept overnight at 4° C. before addition of NaCl. PBS, untreated (unboiled) sample in PBS; Boiled PBS, Boiled nanoparticules in PBS; Boiled [NaCl] 1M, Boiled nanoparticles incubated 8 h in 1M NaCl before spectrum acquisition. (C) PEPEG-capped nanoparticles in PBS were frozen for 72 h at −20° C. and then thawed at room temperature before addition of NaCl. PBS, untreated (unfrozen) sample in PBS; Frozen PBS, frozen nanoparticules in PBS; Frozen [NaCl] 1M, frozen nanoparticles incubated 8 hrs in 1M NaCl before spectrum acquisition.

1. A nanoparticle conjugate comprising a nanoparticle having one or more compounds attached thereto, wherein at least one of the one or more compounds comprise at least one ethylene glycol unit.

2. A nanoparticle conjugate as claimed in claim 1 comprising a nanoparticle having one or more peptide-ol compound and one or more polyethylene glycol (PEG) compounds.

3. A nanoparticle conjugate as claimed in claim 2, wherein the peptide-ol compound comprises one or more cysteine residues.

4. A nanoparticle conjugate as claimed in claim 3, wherein the peptide-ol compound is attached to the nanoparticle by means of one or more cysteine residues.

5. A nanoparticle conjugate as claimed in claim 4, wherein the one or more cysteine residues are located at one end of the peptide-ol.

6. A nanoparticle conjugate as claimed in claim 3, wherein the cysteine residue is attached to the nanoparticle by means of its thiol and/or amino group.

7. A nanoparticle conjugate as claimed in claim 2, wherein the peptide-ol compound is a pentapeptide-ol.

8. A nanoparticle conjugate as claimed in claim 2, wherein the PEG compound comprises a thiolated alkyl.

9. A nanoparticle conjugate as claimed in claim 8, wherein the length of the alkyl chain is substantially similar to the length of the peptide-ol compound.

10. A nanoparticle conjugate as claimed in claim 2, wherein the one or more peptide-ol compounds is selected from the group consisting of: H₂N-Cysteine-Alanine-Leucine-Asparagine-Asparaginol (CALNN(ol)), H₂N-Cysteine-Cysteine-Alanine-Leucine-Asparagine-Asparaginol-(CCALNN(ol)), H₂N-Cysteine-Valine-Valine-Valine-Threoninol-(CVVVT(ol)), H₂N-Cysteine-Cysteine-Valine-Valine-Valine-Threoninol (CCVVVT(ol)), H₂N-Cysteine-Serine-Serine-Serine-Serinol, (CSSSS(ol)), H₂N-Cysteine-Cysteine-Serine-Serine-Serine-Serinol, (CCSSSS(ol)) and the reverse of the above sequences where the N-terminal amino acid has its amino group replaced by an alcohol, and mixtures thereof.

11. A nanoparticle conjugate as claimed in claim 1, wherein one or more compounds are attached to the surface of the nanoparticle so as to provide a shell.

12. A nanoparticle conjugate as claimed in claim 11, wherein a plurality of peptide-ol compounds and a plurality of PEG compounds are attached to the surface of the nanoparticle so as to provide a shell.

13. A nanoparticle conjugate as claimed in claim 12, wherein the peptide-ol compounds and PEG compounds are present in ratios between 95:5 and 5:95 (mole/mole).

14-16. (canceled)

17. A nanoparticle conjugate as claimed in claim 1, wherein the one or more compound comprises a peptide ethylene glycol.

18. A nanoparticle conjugate as claimed in claim 1, wherein the nanoparticle is produced from a metal/metallic material, a magnetic material and/or a semi-conducting material.

19. A nanoparticle conjugate as claimed in claim 18 wherein the nanoparticle comprises or consists of a superparamagnetic particle or a quantum dot.

20. A nanoparticle conjugate as claimed in claim 1, wherein nanoparticle is produced from gold, silver, cobalt, nickel, platinum, cadmium selenide or zinc sulphide.

21. A nanoparticle conjugate as claimed in claim 1 further comprising one or more ligands.

22. (canceled)

23. A nanoparticle conjugate as claimed in claim 1, wherein the nanoparticle conjugate further comprises an identification means attached to the one or more compounds.

24. (canceled)

25. A nanoparticle conjugate as claimed in claim 2, wherein the nanoparticle conjugate comprises different subgroups of peptide-ols.

26. A nanoparticle conjugate as claimed in claim 25, wherein different ligands and optionally different identification means and/or functional groups are attached to different subgroups of peptide-ols.

27. A nanoparticle conjugate as claimed in claim 1, wherein the nanoparticle conjugate is capable of being conjugated to at least one other nanoparticle conjugate or is conjugated to at least one other nanoparticle conjugate to form nanoparticle conjugate assemblies.

28. A nanoparticle conjugate as claimed in claim 26, wherein the identification means and/or functionalised group and/or ligand is selected from the group consisting of: biotin and/or avidin, streptavidin, streptactin, Histidine tags, NTA, radio active labels, antigens, epitopes or parts of epitopes, antibodies, fluorochromes, nucleic acids, recognition sequences, enzymes, antibodies, peptide-ols, peptides, proteins, receptors and target molecules, saccharides, polysaccharides and lipids.

29. (canceled)

30. A nanoparticle conjugate as claimed in claim 1, wherein the nanoparticle has a diameter in the range of 1-100 nm.

31-32. (canceled)

33. A nanoparticle as claimed in claim 1 wherein the compound comprising at least one ethylene glycol unit has the formula:
SH—(CH2)n-EGx
Where:
n=1 to 20;
EG=ethylene glycol unit; and
x=1 to 10

34. A nanoparticle as claimed in claim 1 wherein the compound comprising at least one ethylene glycol unit comprises CVVVT-EGn-ol
Where EG=ethylene glycol unit; and
n=1 to 10.

35. A method of producing a nanoparticle conjugate as described in claim 2 by incubating in water, a nanoparticle solution with a mixture of peptide-ols in a phosphate-buffered saline and PEG.

36. A method as claimed in claim 33, wherein one or more ligands and optionally one or more identification means and/or functional groups are conjugated to the peptide-ol and/or PEG prior to incubation with the nanoparticle or during the course of the incubation.

37. A method as claimed in claim 35, which further comprises freeze drying the nanoparticle conjugate, or boiling or autoclaving the nanoparticle conjugate.

38-40. (canceled)

41. The method of claim 35 comprising a) solubilizing nanoparticle in aqueous buffer, and b) centrifuging.

42. A method as claimed in claim 41 wherein centrifuging step comprises Nanosep centrifugal ultrafiltration.

43. A method as claimed in claim 41 wherein the nanoparticle is a quantum dot.
